(19) United States
(12) Patent Application Publication
(10) Pub. No.: US 2023/0207084 A1
(43) Pub. Date: Jun. 29, 2023

(54) PHARMACEUTICAL COMBINATION PARAMETER ESTIMATION VIA MODEL SURROGATE

(71) Applicant: International Business Machines Corporation, Armonk, NY (US)

(72) Inventors: James R. Kozloski, New Fairfield, CT (US); Tim Rumbell, Raleigh, NC (US); VIATCHESLAV GUREV, Bedford Hills, NY (US)

(21) Appl. No.: 17/560,734

(22) Filed: Dec. 23, 2021

(51) Int. Cl. G16H 20/10; G16C 20/70; G16C 20/30

(52) U.S. Cl. CPC G16H 20/10; G16C 20/70; G16C 20/30

(57) ABSTRACT

A system may include a memory and a processor in communication with the memory. The processor may be configured to perform operations. The operations may include training a cr-GAN model with a first data set of a first pharmaceutical and a second data set of a second pharmaceutical. The operations may include conditioning the cr-GAN model with at least one conditional variable and generating, with the cr-GAN model, patient parameters. The operations may include replicating a set of patient data of a patient with the patient parameters and calculating dosage data with the patient parameters based on a therapeutic target. The operations may include displaying the dosage data to a user.

### BACKGROUND

The present disclosure relates to pharmaceutical combination delivery and more specifically to delivery parameters of pharmaceutical combinations.

Combining pharmaceutical agents may address multiple ailments simultaneously or may be used to treat a single ailment with improved efficacy levels. Use of multiple pharmaceutical agents may result in pharmokinetic interplay of the reagents such that the pharmaceutical agents interact with each other and enter the system differently than if independently administered and, thus, such an administration of the agents may impact the results. Pharmokinetic interplay may be detrimental or beneficial, and the detriment or benefit derived may be a function of the doses of the pharmaceuticals administered. Dosage of one or more pharmaceutical agents may involve several variables.

### SUMMARY

Embodiments of the present disclosure include a system, method, and computer program product for pharmaceutical combination delivery parameters.

A system in accordance with the present disclosure may include a memory and a processor in communication with the memory. The processor may be configured to perform operations. The operations may include training a cr-GAN model with a first data set of a first pharmaceutical and a second data set of a second pharmaceutical. The operations may include conditioning the cr-GAN model with at least one conditional variable and generating, with the cr-GAN model, patient parameters. The operations may include replicating a set of patient data of a patient with the patient parameters and calculating dosage data with the patient parameters based on a therapeutic target. The operations may include displaying the dosage data to a user.

The above summary is not intended to describe each illustrated embodiment or every implementation of the disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 illustrates a system in accordance with some embodiments of the present disclosure.

FIG. 2 depicts a pharmacokinetic conditioned regularized generative adversarial network use case progression diagram in accordance with some embodiments of the present disclosure.

FIG. 3 illustrates a graph set in accordance with some embodiments of the present disclosure.

FIG. 4 depicts a graph set in accordance with some embodiments of the present disclosure.

FIG. 5 illustrates a graph in accordance with some embodiments of the present disclosure.

FIG. 6 depicts a graph set in accordance with some embodiments of the present disclosure.

FIG. 7 illustrates a method in accordance with some embodiments of the present disclosure.

FIG. 8 illustrates a cloud computing environment, in accordance with embodiments of the present disclosure.

FIG. 9 depicts abstraction model layers, in accordance with embodiments of the present disclosure.

FIG. 10 illustrates a high-level block diagram of an example computer system that may be used in implementing one or more of the methods, tools, and modules, and any related functions, described herein, in accordance with embodiments of the present disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to pharmaceutical combination delivery and more specifically to delivery parameters of pharmaceutical combinations.

Dose selection for the treatment of cancerous tumors with multiple chemotherapeutic agents may involve identifying tumor-size-dependent efficacy isoboles for various pharmaceutical combinations available from a standard pharmacopeia or during clinical trials involving various pharmaceutical combinations. Pharmaceutical combinations may be desirable because combining pharmaceuticals at sub-toxic doses may achieve efficacy levels exceeding that which an isolated pharmaceutical may achieve and/or efficacy levels which would require one or more toxic doses of an individual pharmaceutical to achieve.

Isoboles may be used to represent nonlinear manifolds of pharmaceutical combinations over which efficacy is constant. Dosing may aim to maximize a synchronous concentration of the desired pharmaceutical combination at the tumor, thereby accessing the optimal isobole for a therapeutic target such as a reduction in tumor size.

In accordance with the present disclosure, a conditioned regularized generative adversarial network (cr-GAN) architecture may be used for generating mechanistic model M parameter samples x_{g} that may produce outputs y_{g} coherent with a set of observed data y. The cr-GAN generator may be conditioned on auxiliary observed data a that is not directly accessible to the mechanistic model. Such an implicit generative model may be formulated as:
Given P_{X}, Q_{Y,A}, M
Minimize D(P_{X}∥Q_{X}_{_{g}})

Subject to supp(X_{g})⊆supp(X) and D(Q_{Y,A}∥Q_{Y}_{_{g}}_{,A})=0 Eq. 1
Where [y_{g}, a]=[M(x_{g}), a]˜Q_{Y}_{_{g}}_{,A} and [x_{g}, a]˜Q_{X}_{_{g}}_{,A}

where joint distributions Q_{X,A}, Q_{X}_{_{g}}_{,A} and Q_{Y,A} have marginals Q_{X}, Q_{X}_{_{g}}, and Q_{Y}, respectively, and D(·∥·) is an f-divergence measure such a Jensen-Shannon divergence (JSD). Eq. 1 may be solved using a GAN by minimizing divergence D(P_{X}∥Q_{X}_{_{g}}) between a given prior P_{X} and generated model parameters Q_{X}_{_{g}} over network parameters θ in the generator:

z˜P_{Z,a}˜Q_{A,x}_{_{g}}=G_{θ}(z,a)˜Q_{X}_{_{g}} Eq. 2

where P_{Z} is a Gaussian base distribution, P_{X} is the prior distribution of model parameters, and Q_{A} is the marginal of Q_{Y,A} for auxiliary variable A. Simultaneously, D(Q_{Y,A}∥Q_{Y}_{_{g}}_{,A}) over θ may be minimized in the generator:

[y_{g},a]=[M(G_{θ}(z,a)),a]˜Q_{Y}_{_{g}}_{,A} Eq. 3

To approximate D(Q_{Y,A}∥Q_{Y}_{_{g}}_{,A})=0 while minimizing D(P_{X}∥Q_{X}_{_{g}}), the two objectives may be incorporated as separate discriminators with a weighted sum loss such that the weight for the generator loss due to discriminator D_{X} is smaller than that for D_{Y}. Auxiliary variable A may be incorporated as a conditioning variable in G and D_{Y}.

In accordance with the present disclosure, such a GAN architecture may consist of one generator and two discriminators and a reconstruction network that recreates Z from the output of G and a function representing the mechanistic model M. Each of the networks in such a GAN architecture may be a feedforward neural network such as one described by Table 1.

TABLE 1
Details of Neural Networks Used in a GAN Architecture

| | Hidden | Nodes | Dropout | Activation |
|---|---|---|---|---|
| Network | Layers | Per Layer | Rate | Function |
| D_{X} | 8 | 80 | 0.0 | RELU |
| D_{Y} | 8 | 130 | 0.01 | RELU |
| G | 8 | 80 | 0.0 | RELU |
| R | 8 | 180 | 0.0 | RELU |

Discriminator D_{Y} distinguishes between samples from the joint distribution Q_{Y,A} and samples generated by the generator G forwarded through the mechanistic model and augmented with the conditioning variable A. The standard conditional loss L_{D}_{_{Y}} of the discriminator D_{Y} may be described as:

L_{D}_{_{Y}}= [symbol] _{y,a˜Q}_{_{Y,A}} log[D_{Y}(y,a)]+ [symbol] _{z˜P}_{_{Z,a}}_{˜Q}_{_{A}} log[1−D_{Y}(M(G(z,a)),a)] Eq. 4

The standard conditional loss D_{Y} may be maximized. Discriminator D_{X} distinguishes between samples from the prior over mechanistic parameters P_{X} and samples generated by G. The standard loss L_{D}_{_{X}} may be expressed as:

L_{D}_{_{X}}= [symbol] _{x˜P}_{ₓ} log[D_{X}(x)]+ [symbol] _{z˜P}_{_{Z}} log[1−D_{X}(G(z))] Eq. 5

The standard conditional loss D_{Y} may be maximized. The reconstruction network R aims to reproduce the original base distribution Z from samples generated by G. The squared loss L_{R} may be described as:

L_{R}= [symbol] _{z˜P}_{_{z,a}}_{˜Q}_{_{A}}[z−R(G(z,a))]² Eq. 6

The squared loss L_{R} may be minimized.

The generator network G generates mechanistic parameter sets from the base variable Z, augmented with the auxiliary observed data a. The weighted sum loss L_{G} may be expressed as:

L_{G}=w_{Y}L_{D}_{_{Y}}+w_{X}L_{D}_{_{X}}+w_{R}L_{R} Eq. 7

The weighted sum loss L_{G} may be minimized where w_{Y}=1.0, wₓ=0.1, and w_{R}=1.0.

For results shown here, the Adam optimizer used had step size of 0.00001 for G and R, 0.00002 for D_{X}, and 0.00001 for D_{Y}. The β₁ and β₂ parameters of the Adam optimizer were set to default values of 0.9 and 0.999, respectively. Mini-batch size was 100. Training was performed in two stages: first, G, R, and D_{X} were trained together with w_{X}=1.0 and the L_{D}_{_{Y}} term removed in Eq. 7 for 100 epochs to initialize G by minimizing D(P_{X}∥Q_{X}_{_{g}}); second, the full GAN was trained for 300 epochs on a dataset y, a˜Q_{Y,A} of 10,000 samples.

Divergence between distributions may be tested with JSD and approximated using density ratio estimation with a binary classifier to approximate the KL divergence measure from samples. In this approach, JSD may be estimated using a classifier network trained to distinguish samples from the two distributions.

Due to pharmacokinetics, synchronous concentration of a combination may depend on the specific pharmaceuticals desired, the time of administration of the pharmaceuticals, and the size of the tumor. An appropriate model of these pharmacokinetics for each pharmaceutical under each of these conditional variables may be used to identify optimal delivery parameters. A cr-GAN may be used to sample pharmacokinetic (PK) model parameters conditioned by pharmaceutical identity and tumor size of a patient to recommend pharmaceutical combination therapies to access the desired isobole with the highest probability. In some embodiments, the present disclosure may use an algorithmic core of a cr-GAN inverse PK model to such an effect.

In accordance with the present disclosure, a cr-GAN may be trained to sample from the parameter space of a PK model for each pharmaceutical in the desired combination therapy. Conditional variables specific to the condition may be used; for example, in the treatment of cancer, an initial tumor size may be used as one conditional variable and another conditional variable may be an observed baseline change in the tumor size given an identified dose of an identified pharmaceutical.

The trained cr-GAN may be used to sample parameters of the PK model, given conditional variables and a therapeutic target, to construct an isobole for a particular pharmaceutical combination. A therapeutic target may be, for example, a specific tumor size reduction (e.g., 20% reduction in tumor size within six months). The isobole constructed with the cr-GAN samples, the PK model, and patient response data may be, for example, a 95% isobole such that the isobole identifies doses at which the same or a similar therapeutic target was achieved in 95% of patients with similar characteristics. Similar characteristics may be, for example, patient demographics, health history, illness type, or other factors which may impact the efficacy of the use of one or more desired pharmaceuticals.

A treatment opportunity window may be identified in the isobole, and a combination dosing therapy may be selected from the treatment opportunity window to maximize dose efficiency while simultaneously minimizing dose toxicity of the pharmaceutical combination.

In some embodiments, an additional cr-GAN may be trained with efficacy data concerning pharmaceutical combinations. Such a pharmaceutical combination cr-GAN may be used to parameterize a nonlinear model of efficacy given an expected concentration of the pharmaceutical at the treatment site (e.g., the tumor). The parameterized nonlinear efficacy model may be used to augment or otherwise modify the isobole construction algorithm developed by the first trained cr-GAN to improve the isobole (e.g., more accurately estimate a 95% isobole).

In some embodiments of the present disclosure, a cr-GAN for constrained optimization may be applied to samples from a distribution of parameters. The parameters may replicate a set of patient data including information such as, for example, absorption, distribution, metabolism, and/or excretion of pharmaceuticals; such data may describe pharmaceutical impact on the patient independently (e.g., when only one pharmaceutical is used in the patient) or in combination (e.g., how multiple pharmaceuticals react with a patient when administered together).

The parameters may be used to parameterize a PK model of multiple pharmaceuticals subject to conditioning variables. Conditioning variables may include, for example, a baseline patient state (e.g., in cancer treatment, initial tumor size), an observed change from the baseline (e.g., the change of the tumor size since the initial measurement), and a therapeutic target (e.g., a 25% reduction in tumor size within one year). The trained cr-GAN may determine pharmaceutical dosage data necessary to achieve the therapeutic target.

In some embodiments, the pharmaceutical dosage data may be used to determine an isobole contour plot of pharmaceutical efficacy. Pharmaceutical efficacy information may be plotted on the isobole contour plot such that a user may identify a desired efficacy (e.g., an efficacy on the isobole contour plot within a treatment opportunity window to maximize effectiveness and minimize toxicity) as a contour on the isobole contour plot.

In some embodiments, the isobole contour plot may be refined using a second cr-GAN. The second cr-GAN may be trained to sample from parameters of a model (such as a quantitative systems pharmacology model) capable of mapping pharmaceutical doses to a given distribution of efficacy measures and tumor sizes associated with each efficacy measure.

In some embodiments, the pharmaceutical combination may be chosen from a point on the desired efficacy isobole that falls within the treatment opportunity window such that the dosage avoids toxic effects of the pharmaceuticals while maintaining efficacy.

A system in accordance with the present disclosure may include a memory and a processor in communication with the memory. The processor may be configured to perform operations. The operations may include training a cr-GAN model with a first data set of a first pharmaceutical and a second data set of a second pharmaceutical. The operations may include conditioning the cr-GAN model with at least one conditional variable and generating, with the cr-GAN model, patient parameters. The operations may include replicating a set of patient data of a patient with the patient parameters and calculating dosage data with the patient parameters based on a therapeutic target. The operations may include displaying the dosage data to a user.

In some embodiments of the present disclosure, the operations may include generating the first data set with a pharmacokinetic model.

In some embodiments of the present disclosure, the operations may include generating the first data set with a quantitative systems pharmacology model.

In some embodiments of the present disclosure, the operations may include describing an interaction between the first pharmaceutical and the patient in the patient data. In some embodiments, the interaction may describe a first absorption, a first distribution, a first metabolism, and/or a first excretion.

In some embodiments of the present disclosure, the at least one conditional variable may include at least one of baseline tumor size, current tumor size, observed tumor size change, and observed tumor size change rate.

In some embodiments of the present disclosure, the operations may include determining an isobole contour plot based on the dosage data and displaying calculated efficacies of the first pharmaceutical and the second pharmaceutical on the isobole contour plot.

In some embodiments of the present disclosure, the operations may include training a second cr-GAN model with a distribution of efficacy measures and tumor sizes associated with the efficacy measures, sampling from the dosage data with the second cr-GAN to parameterize a quantitative systems pharmacology model to identify a recommended dosage, and including the recommended dosage in the dosage data.

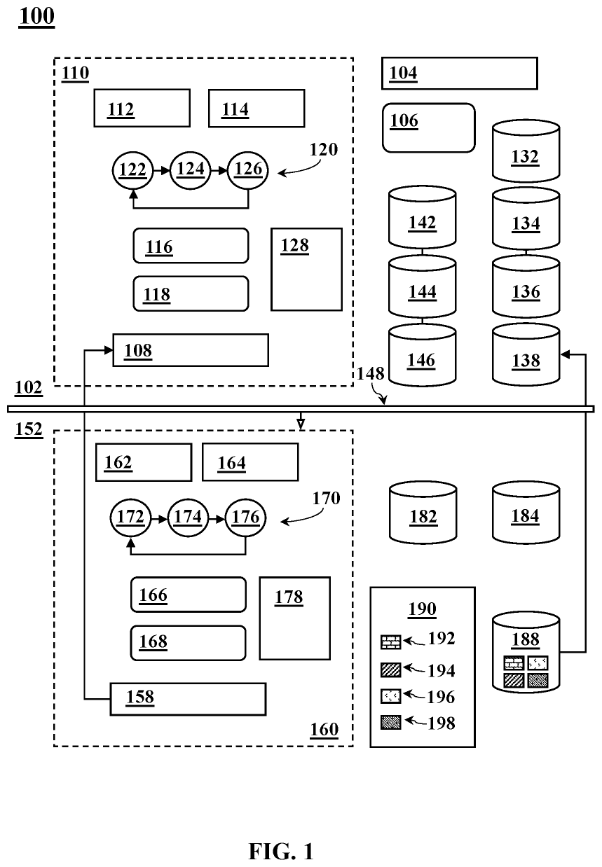

FIG. 1 illustrates a system 100 in accordance with some embodiments of the present disclosure. The system 100 may include a MaaS deployment log 102 and a flow manager 104. The system 100 may include a research side 102 and a deployment side 152 separated by a firewall 148. In some embodiments, the firewall 148 may be a network address translation (NAT) component or other communication interface mechanism.

The system 100 may include several databases on the research side 102 of the firewall 148 including, for example, a model simulation data database 132, a GAN graph library database 134, an inverse surrogate library database 136, a synthetic test data database 138, a mechanistic model package database 142, a model optimizer library database 144, and a forward surrogate library database 146.

The system 100 may include several databases on the deployment side 152 such as, for example, a mechanical model library database 182, a statistical model database 184, and a proprietary information database 188. The proprietary information database 188 may contain device and experiment 190 information such as, for example, mechanism prior data 192, pharmaceutical data 194, conditioning data 196, and target data 198. The proprietary information database 188 may be in communication with the synthetic test data database 138.

The research side 102 of the system 100 may include a research virtual machine 110 which houses engine graph data 112, GAN graph data 114, and a cloud deployment API 128. The research virtual machine 110 may include a model generation process 120 which may include simulation 122, validation 124, and parameterization 126 of a model. The research virtual machine 110 may include a stateless handler 116, a stateful partition processor 118, and an analytics event log 108.

The analytics event log 108 in the research virtual machine 110 may be in communication with an analytics event log 158 in a deployment virtual machine 160. The deployment virtual machine 160 may also include engine graph data 162, GAN graph data 164, and a cloud deployment API 178. The deployment virtual machine 160 may further include a model generation process 170 which may include simulation 172, validation 174, and parameterization 176 of a model. The deployment virtual machine 160 may include a stateless handler 166, a stateful partition processor 168.

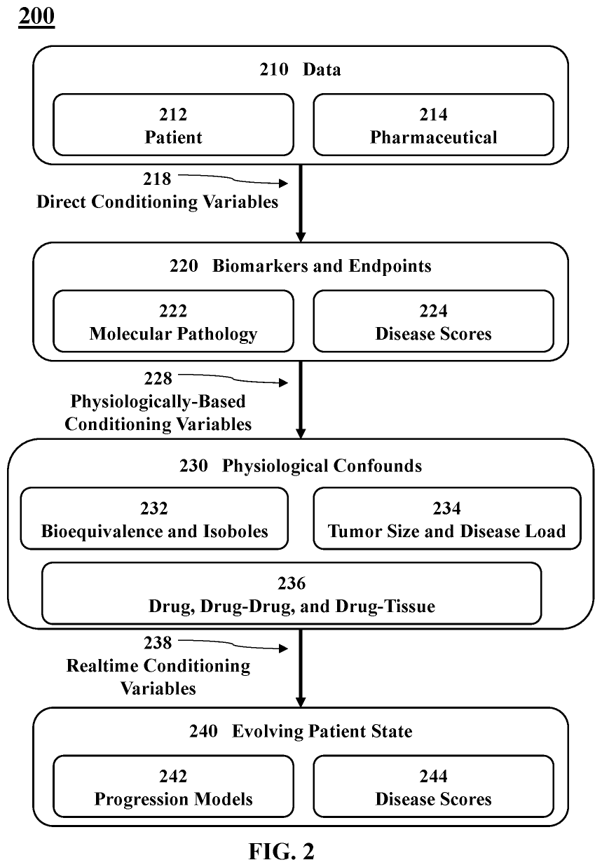

FIG. 2 depicts a PK cr-GAN use case progression diagram 200 in accordance with some embodiments of the present disclosure. Data 210 is used to identify biomarkers and endpoints 220 which is used to identify physiological confounds 230 which is used to monitor an evolving patient state 240.

The data 210 may include patient data 212 and pharmaceutical data 214. Direct conditioning variables 218 may be identified in the data; the direct conditioning variables 218 may be used in the identification of the biomarkers and endpoints 220.

The biomarkers and endpoints 220 may include molecular pathology 222 and disease scores 224. The physiologically-based conditioning variables 228 may be identified in the data; the physiologically-based conditioning variables 228 may be used for the identification of the physiological confounds 230.

The physiological confounds 230 may include bioequivalence and isoboles 232, tumor size and disease load 234, and pharmaceutical, pharmaceutical-pharmaceutical, and pharmaceutical-tissue 236 information. Realtime conditioning variables 238 may be identified in the physiological confounds 230; the real-time conditioning variables 238 may be used for monitoring an evolving patient state 240.

The evolving patient state 240 may be monitored using progression models 242 and disease scores 244. Other mechanisms may be used for identifying, monitoring, assessing, and quantifying the evolving patient state 240. In some embodiments, the evolving patient state 240 may be communicated to a user such as a care provider.

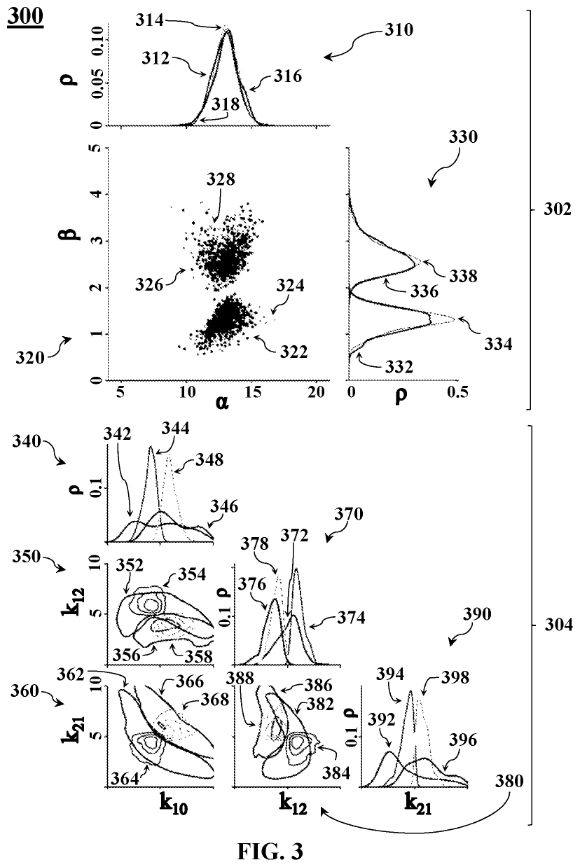

FIG. 3 illustrates a graph set 300 in accordance with some embodiments of the present disclosure. The graph set 300 displays samples from cr-GAN simulations using two distinct, disjointed subsets of a dataset. The graph set 300 includes a set of features graphs 302 and a set of parameters graphs 304.

The features graphs 302 include a scatterplot 320 of a beta (β) feature distribution plotted against an alpha (α) feature distribution. The scatterplot 320 includes a first set of cr-GAN samples 322 corresponding to a first set of target data 324. The scatterplot 320 includes a second set of cr-GAN samples 326 corresponding to a second set of target data 328.

The features graphs 302 include a p-a line graph 310 of density (ρ) tracked against the alpha (α) feature. The ρ-α line graph 310 includes a first set of cr-GAN samples 312 corresponding to a first set of target data 314 and a second set of cr-GAN samples 316 corresponding to a second set of target data 318.

The features graphs 302 include a ρ-β line graph 330 of density (ρ) tracked against the beta (β) feature. The ρ-β line graph 330 includes a first set of cr-GAN samples 332 corresponding to a first set of target data 334 and a second set of cr-GAN samples 336 corresponding to a second set of target data 338.

The parameters graphs 304 include line graphs and contour graphs tracking parameters against other parameters and/or density. The parameters graphs 304 include a ρ-k₁₀ line graph 340 of density (ρ) tracked against parameter k₁₀. The ρ-k₁₀ line graph 340 includes a first set of cr-GAN samples 342 corresponding to a first set of true parameters 344 and a second set of cr-GAN samples 346 corresponding to a second set of true parameters 348.

The parameters graphs 304 include a k₁₂-k₁₀ contour graph 350 of parameter k₁₂ tracked against parameter k₁₀. The k₁₂-k₁₀ contour graph 350 includes a first set of cr-GAN samples 352 corresponding to a first set of true parameters 354 and a second set of cr-GAN samples 356 corresponding to a second set of true parameters 358.

The parameters graphs 304 include a k₂₁-k₁₀ contour graph 360 of parameter k₂₁ tracked against parameter k₁₀. The k₂₁-k₁₀ contour graph 360 includes a first set of cr-GAN samples 362 corresponding to a first set of true parameters 364 and a second set of cr-GAN samples 366 corresponding to a second set of true parameters 368.

The parameters graphs 304 include a ρ-k₁₂ line graph 370 of density (ρ) tracked against parameter k₁₂. The ρ-k₁₂ line graph 370 includes a first set of cr-GAN samples 372 corresponding to a first set of true parameters 374 and a second set of cr-GAN samples 376 corresponding to a second set of true parameters 378.

The parameters graphs 304 include a k₂₁-k₁₂ contour graph 380 of parameter k₂₁ tracked against parameter k₁₂. The k₂₁-k₁₂ contour graph 380 includes a first set of cr-GAN samples 382 corresponding to a first set of true parameters 384 and a second set of cr-GAN samples 386 corresponding to a second set of true parameters 388.

The parameters graphs 304 include a ρ-k₁₀ line graph 390 of density (ρ) tracked against parameter k₂₁. The ρ-k₂₁ line graph 390 includes a first set of cr-GAN samples 392 corresponding to a first set of true parameters 394 and a second set of cr-GAN samples 396 corresponding to a second set of true parameters 398.

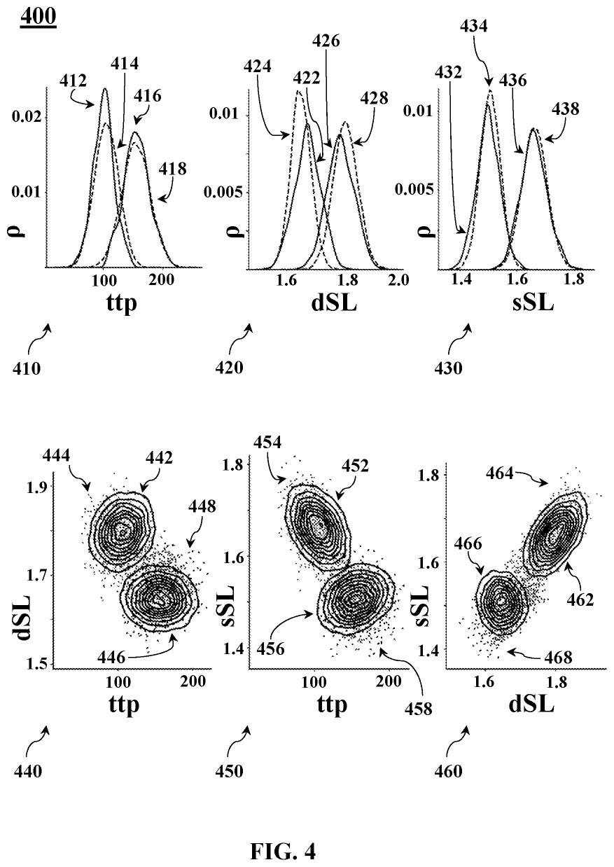

FIG. 4 depicts a graph set 400 in accordance with some embodiments of the present disclosure. The graph set 400 tracks density (ρ), resting sarcomere length (dSL), sarcomere length at maximum contraction (sSL), and time to peak contraction (ttp) for inferred distributions and corresponding target distributions.

The first graph 410 of the graph set 400 tracks density (ρ) as it relates to time to peak contraction (ttp). The first graph 410 includes a first ρ-ttp inferred distribution 412 and a corresponding first ρ-ttp target distribution 414. The first graph 410 includes a second ρ-ttp inferred distribution 416 and a corresponding second ρ-ttp target distribution 418.

The second graph 420 of the graph set 400 tracks density (ρ) as it relates to resting sarcomere length (dSL). The second graph 420 includes a first ρ-dSL inferred distribution 422 and a corresponding first ρ-dSL target distribution 424. The second graph 420 includes a second ρ-dSL inferred distribution 426 and a corresponding second ρ-dSL target distribution 428.

The third graph 430 of the graph set 400 tracks density (ρ) as it relates to sarcomere length at maximum contraction (sSL). The third graph 430 includes a first ρ-sSL inferred distribution 432 and a corresponding first ρ-sSL target distribution 434. The third graph 430 includes a second ρ-sSL inferred distribution 436 and a corresponding second ρ-sSL target distribution 438.

The fourth graph 440 of the graph set 400 tracks resting sarcomere length (dSL) as it relates to time to peak contraction (ttp). The fourth graph 440 includes a first set of dSL-ttp inferred distribution samples 444 (scatterplot) and a corresponding first dSL-ttp target distribution 442 (contour lines). The fourth graph 440 includes a second set of dSL-ttp inferred distribution samples 448 (scatterplot) and a corresponding second dSL-ttp target distribution 446 (contour lines).

The fifth graph 450 of the graph set 400 tracks sarcomere length at maximum contraction (sSL) as it relates time to peak contraction (ttp). The fifth graph 450 includes a first set of sSL-ttp inferred distribution samples 454 (scatterplot) and a corresponding first sSL-ttp target distribution 452 (contour lines). The fifth graph 450 includes a second set of sSL-ttp inferred distribution samples 458 (scatterplot) and a corresponding second sSL-ttp target distribution 456 (contour lines).

The sixth graph 460 of the graph set 400 tracks sarcomere length at maximum contraction (sSL) as it relates to resting sarcomere length (dSL). The sixth graph 460 includes a first set of sSL-dSL inferred distribution samples 464 (scatterplot) and a corresponding first sSL-dSL target distribution 462 (contour lines). The sixth graph 460 includes a second set of sSL-dSL inferred distribution samples 468 (scatterplot) and a corresponding second sSL-dSL target distribution 466 (contour lines).

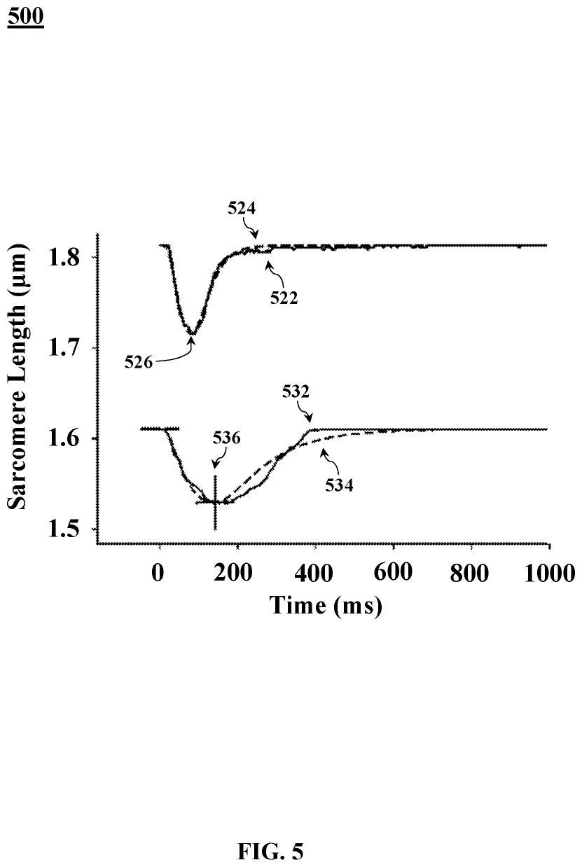

FIG. 5 illustrates a graph 500 in accordance with some embodiments of the present disclosure. The graph 500 tracks sarcomere length in micrometers over time in milliseconds to identify time to peak contraction (ttp) and the rate of relaxation (k2) after achieving peak contraction for inferred distributions (depicted in graph 500 as solid lines) and target distributions (depicted in graph 500 as dashed lines). The graph 500 includes simulated model projections tracked with corresponding experimental data.

The graph 500 includes a first inferred distribution 522, a first target distribution 524, and a first peak contraction point 526. The first inferred distribution 522 closely models the first target distribution 524. The graph 500 includes a second inferred distribution 532, a second target distribution 534, and a second peak contraction point 536. The second inferred distribution 532 closely models the second target distribution 534.

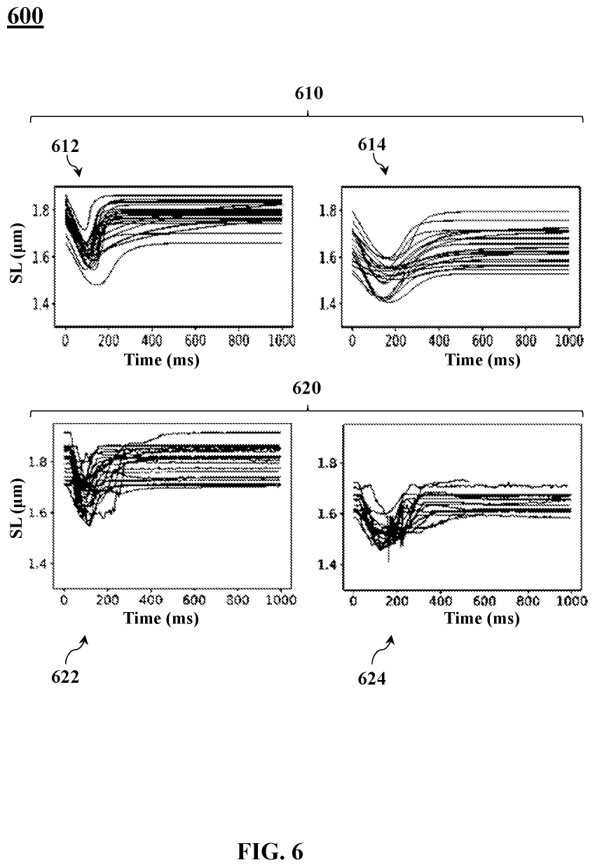

FIG. 6 depicts a data graph set 600 in accordance with some embodiments of the present disclosure. The graph set 600 includes model simulation data graphs 610 and corresponding experimental data graphs 620. Each graph in the data graph set 600 tracks sarcomere length in micrometers over time in milliseconds.

The model simulation data graphs 610 include a control model simulation graph 612 predicted based on control data. The control data corresponds to a control dataset captured in the control experimental dataset graph 622 of the experimental data graphs 620.

The model simulation data graphs 610 include a model simulation graph 614 predicted based on Omecamtiv Mecarbil (OM) data. The experimental data corresponds to an uncontrolled experimental OM dataset captured in the experimental dataset graph 624 of the experimental data graphs 620.

A computer-implemented method in accordance with the present disclosure may include training a cr-GAN model with a first data set of a first pharmaceutical and a second data set of a second pharmaceutical. The method may include conditioning the cr-GAN model with at least one conditional variable and generating, with the cr-GAN model, patient parameters. The method may include replicating a set of patient data of a patient with the patient parameters and calculating dosage data with the patient parameters based on a therapeutic target. The method may include displaying the dosage data to a user.

In some embodiments of the present disclosure, the method may include generating the first data set with a pharmacokinetic model.

In some embodiments of the present disclosure, the method may include generating the first data set with a quantitative systems pharmacology model.

In some embodiments of the present disclosure, the method may include describing an interaction between the first pharmaceutical and the patient in the patient data. In some embodiments, the interaction describes a first absorption, a first distribution, a first metabolism, and/or a first excretion.

In some embodiments of the present disclosure, the at least one conditional variable may include at least one of baseline tumor size, current tumor size, observed tumor size change, and observed tumor size change rate.

In some embodiments of the present disclosure, the method may include determining an isobole contour plot based on the dosage data and displaying calculated efficacies of the first pharmaceutical and the second pharmaceutical on the isobole contour plot.

In some embodiments of the present disclosure, the method may include training a second cr-GAN model with a distribution of efficacy measures and tumor sizes associated with the efficacy measures, sampling from the dosage data with the second cr-GAN model to identify a recommended dosage, and including the recommended dosage in the dosage data.

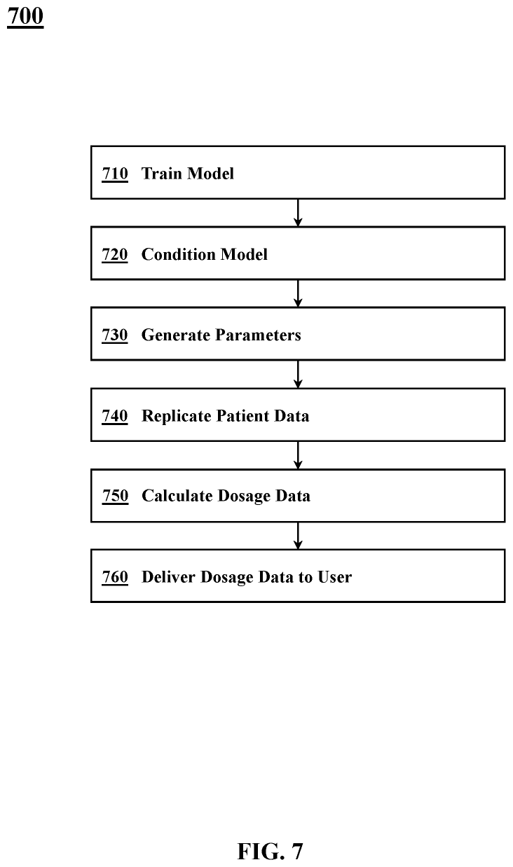

FIG. 7 illustrates a method 700 in accordance with some embodiments of the present disclosure. The method 700 includes training 710 a model, conditioning 720 the model with one or more conditional variables, and generating 730 parameters with the model. The method 700 includes replicating 740 patient data for a patient, calculating 750 dosage data for the patient, and delivering 760 dosage data to a user.

In accordance with the present disclosure, a computer program product may be used to obtain a pharmaceutical combination parameter estimation via model surrogate. A computer program product in accordance with the present disclosure may include a computer readable storage medium having program instructions embodied therewith. The program instructions may be executable by a processor to cause the processor to perform a function. The function may include training a cr-GAN model with a first data set of a first pharmaceutical and a second data set of a second pharmaceutical. The function may include conditioning the cr-GAN model with at least one conditional variable and generating, with the cr-GAN model, patient parameters. The function may include replicating a set of patient data of a patient with the patient parameters and calculating dosage data with the patient parameters based on a therapeutic target. The function may include displaying the dosage data to a user.

In some embodiments of the present disclosure, the function may include generating the first data set with a pharmacokinetic model.

In some embodiments of the present disclosure, the function may include generating the first data set with a quantitative systems pharmacology model.

In some embodiments of the present disclosure, the function may include describing an interaction between the first pharmaceutical and the patient in the patient data. In some embodiments, the interaction describes a first absorption, a first distribution, a first metabolism, and/or a first excretion.

In some embodiments of the present disclosure, the at least one conditional variable may include at least one of baseline tumor size, current tumor size, observed tumor size change, and observed tumor size change rate.

In some embodiments of the present disclosure, the function may include determining an isobole contour plot based on the dosage data and displaying calculated efficacies of the first pharmaceutical and the second pharmaceutical on the isobole contour plot.

In some embodiments of the present disclosure, the function may include training a second cr-GAN model with a distribution of efficacy measures and tumor sizes associated with the efficacy measures, sampling from the dosage data with the second cr-GAN model to identify a recommended dosage, and including the recommended dosage in the dosage data.

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment currently known or that which may be later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model with different physical and virtual resources dynamically assigned and reassigned according to demand. There is a sense of portion independence in that the consumer generally has no control or knowledge over the exact portion of the provided resources but may be able to specify portion at a higher level of abstraction (e.g., country, state, or datacenter).

Rapid elasticity: capabilities can be rapidly and elastically provisioned, in some cases automatically, to quickly scale out and rapidly release to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but the consumer has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software which may include operating systems and applications. The consumer does not manage or control the underlying cloud infrastructure but has control over operating systems, storage, and deployed applications, and the consumer possibly has limited control of select networking components (e.g., host firewalls).

Deployment models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and/or compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

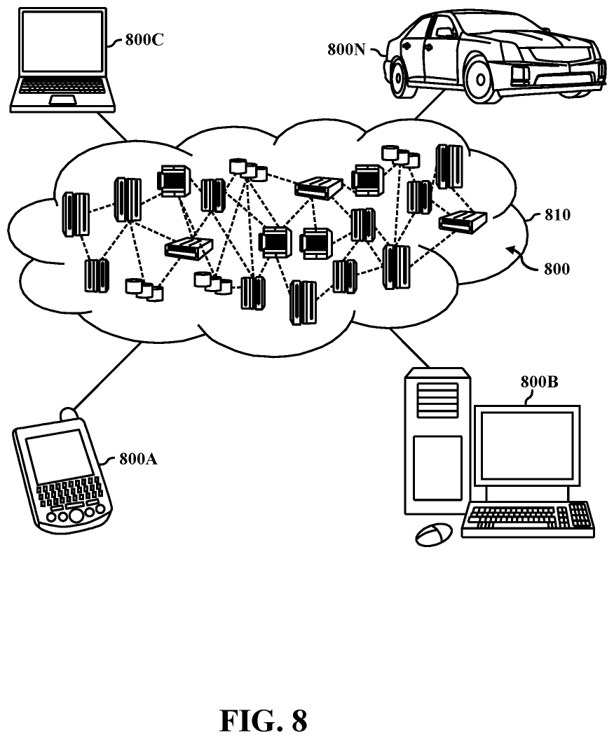

FIG. 8 illustrates a cloud computing environment 810 in accordance with embodiments of the present disclosure. As shown, cloud computing environment 810 includes one or more cloud computing nodes 800 with which local computing devices used by cloud consumers such as, for example, personal digital assistant (PDA) or cellular telephone 800A, desktop computer 800B, laptop computer 800C, and/or automobile computer system 800N may communicate. Nodes 800 may communicate with one another. They may be grouped (not shown) physically or virtually, in one or more networks, such as private, community, public, or hybrid clouds as described hereinabove, or a combination thereof.

This allows cloud computing environment 810 to offer infrastructure, platforms, and/or software as services for which a cloud consumer does not need to maintain resources on a local computing device. It is understood that the types of computing devices 800A-N shown in FIG. 8 are intended to be illustrative only and that computing nodes 800 and cloud computing environment 810 can communicate with any type of computerized device over any type of network and/or network addressable connection (e.g., using a web browser).

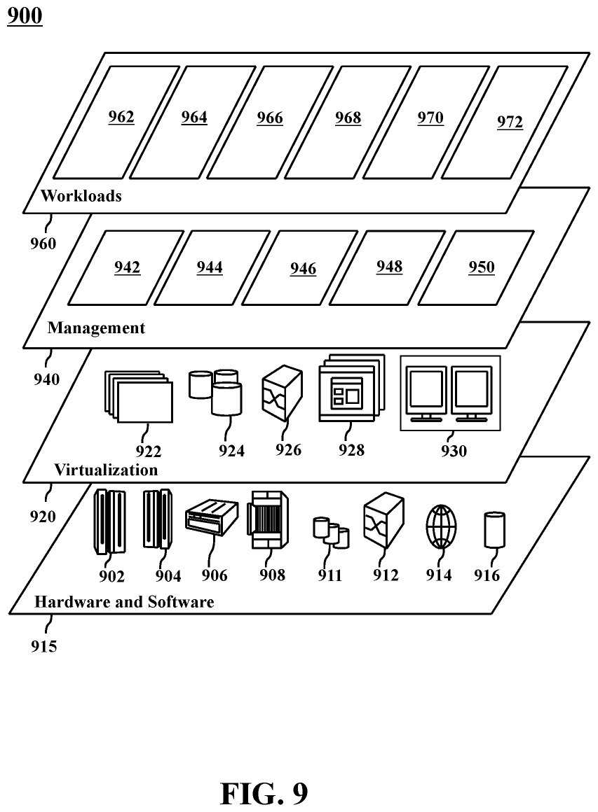

FIG. 9 illustrates abstraction model layers 900 provided by cloud computing environment 810 (FIG. 8) in accordance with embodiments of the present disclosure. It should be understood in advance that the components, layers, and functions shown in FIG. 9 are intended to be illustrative only and embodiments of the disclosure are not limited thereto. As depicted below, the following layers and corresponding functions are provided.

Hardware and software layer 915 includes hardware and software components. Examples of hardware components include: mainframes 902; RISC (Reduced Instruction Set Computer) architecture-based servers 904; servers 906; blade servers 908; storage devices 911; and networks and networking components 912. In some embodiments, software components include network application server software 914 and database software 916.

Virtualization layer 920 provides an abstraction layer from which the following examples of virtual entities may be provided: virtual servers 922; virtual storage 924; virtual networks 926, including virtual private networks; virtual applications and operating systems 928; and virtual clients 930.

In one example, management layer 940 may provide the functions described below. Resource provisioning 942 provides dynamic procurement of computing resources and other resources that are utilized to perform tasks within the cloud computing environment. Metering and pricing 944 provide cost tracking as resources and are utilized within the cloud computing environment as well as billing or invoicing for consumption of these resources. In one example, these resources may include application software licenses. Security provides identity verification for cloud consumers and tasks as well as protection for data and other resources. User portal 946 provides access to the cloud computing environment for consumers and system administrators. Service level management 948 provides cloud computing resource allocation and management such that required service levels are met. Service level agreement (SLA) planning and fulfillment 950 provide pre-arrangement for, and procurement of, cloud computing resources for which a future requirement is anticipated in accordance with an SLA.

Workloads layer 960 provides examples of functionality for which the cloud computing environment may be utilized. Examples of workloads and functions which may be provided from this layer include: mapping and navigation 962; software development and lifecycle management 964; virtual classroom education delivery 966; data analytics processing 968; transaction processing 970; and pharmaceutical combination parameter estimation via model surrogate 972.

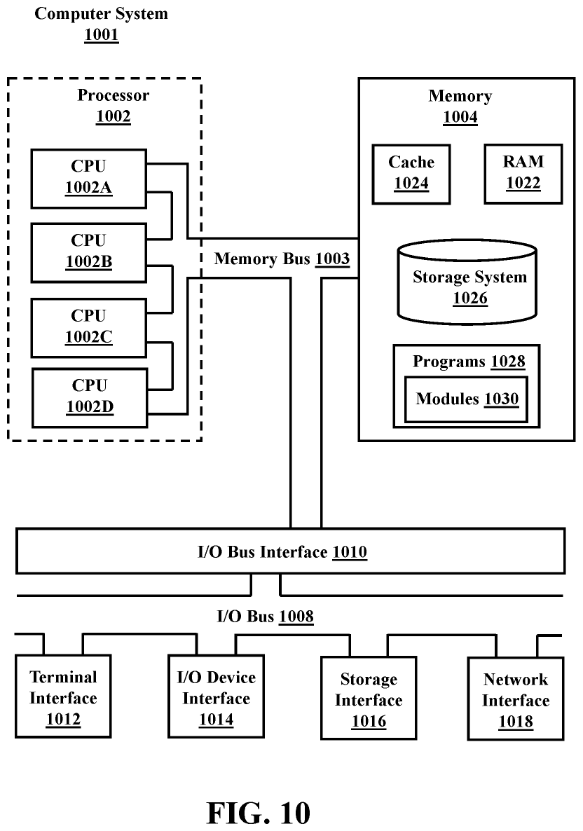

FIG. 10 illustrates a high-level block diagram of an example computer system 1001 that may be used in implementing one or more of the methods, tools, and modules, and any related functions, described herein (e.g., using one or more processor circuits or computer processors of the computer) in accordance with embodiments of the present disclosure. In some embodiments, the major components of the computer system 1001 may comprise a processor 1002 with one or more central processing units (CPUs) 1002A, 1002B, 1002C, and 1002D, a memory subsystem 1004, a terminal interface 1012, a storage interface 1016, an I/O (Input/Output) device interface 1014, and a network interface 1018, all of which may be communicatively coupled, directly or indirectly, for inter-component communication via a memory bus 1003, an I/O bus 1008, and an I/O bus interface unit 1010.

The computer system 1001 may contain one or more general-purpose programmable CPUs 1002A, 1002B, 1002C, and 1002D, herein generically referred to as the CPU 1002. In some embodiments, the computer system 1001 may contain multiple processors typical of a relatively large system; however, in other embodiments, the computer system 1001 may alternatively be a single CPU system. Each CPU 1002 may execute instructions stored in the memory subsystem 1004 and may include one or more levels of on-board cache.

System memory 1004 may include computer system readable media in the form of volatile memory, such as random access memory (RAM) 1022 or cache memory 1024. Computer system 1001 may further include other removable/non-removable, volatile/non-volatile computer system storage media. By way of example only, storage system 1026 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM, or other optical media can be provided. In addition, memory 1004 can include flash memory, e.g., a flash memory stick drive or a flash drive. Memory devices can be connected to memory bus 1003 by one or more data media interfaces. The memory 1004 may include at least one program product having a set (e.g., at least one) of program modules that are configured to carry out the functions of various embodiments.

One or more programs/utilities 1028, each having at least one set of program modules 1030, may be stored in memory 1004. The programs/utilities 1028 may include a hypervisor (also referred to as a virtual machine monitor), one or more operating systems, one or more application programs, other program modules, and program data. Each of the operating systems, one or more application programs, other program modules, and program data, or some combination thereof, may include an implementation of a networking environment. Programs 1028 and/or program modules 1030 generally perform the functions or methodologies of various embodiments.

Although the memory bus 1003 is shown in FIG. 10 as a single bus structure providing a direct communication path among the CPUs 1002, the memory subsystem 1004, and the I/O bus interface 1010, the memory bus 1003 may, in some embodiments, include multiple different buses or communication paths, which may be arranged in any of various forms, such as point-to-point links in hierarchical, star, or web configurations, multiple hierarchical buses, parallel and redundant paths, or any other appropriate type of configuration. Furthermore, while the I/O bus interface 1010 and the I/O bus 1008 are shown as single respective units, the computer system 1001 may, in some embodiments, contain multiple I/O bus interface units 1010, multiple I/O buses 1008, or both. Further, while multiple I/O interface units 1010 are shown, which separate the I/O bus 1008 from various communications paths running to the various I/O devices, in other embodiments some or all of the I/O devices may be connected directly to one or more system I/O buses 1008.

In some embodiments, the computer system 1001 may be a multi-user mainframe computer system, a single-user system, a server computer, or similar device that has little or no direct user interface but receives requests from other computer systems (clients). Further, in some embodiments, the computer system 1001 may be implemented as a desktop computer, portable computer, laptop or notebook computer, tablet computer, pocket computer, telephone, smartphone, network switches or routers, or any other appropriate type of electronic device.

It is noted that FIG. 10 is intended to depict the representative major components of an exemplary computer system 1001. In some embodiments, however, individual components may have greater or lesser complexity than as represented in FIG. 10, components other than or in addition to those shown in FIG. 10 may be present, and the number, type, and configuration of such components may vary.

The present disclosure may be a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide, or other transmission media (e.g., light pulses passing through a fiber-optic cable) or electrical signals transmitted through a wire.

Computer readable program instructions described herein can be downloaded to respective computing/processing devices from a computer readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer, or entirely on a remote computer or server. In the latter scenario, the remote computer may be connected to the user's computer through any type of network, including a local area network (LAN) or a wide area network (WAN) or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus, or other device to produce a computer implemented process such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions which comprises one or more executable instructions for implementing the specified logical function(s). In some alternative implementations, the functions noted in the blocks may occur out of the order noted in the figures. For example, two blocks shown in succession may, in fact, be accomplished as one step, executed concurrently, substantially concurrently, in a partially or wholly temporally overlapping manner, or the blocks may sometimes be executed in the reverse order depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although the present disclosure has been described in terms of specific embodiments, it is anticipated that alterations and modifications thereof will become apparent to the skilled in the art. The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application, or the technical improvement over technologies found in the marketplace or to enable others of ordinary skill in the art to understand the embodiments disclosed herein. Therefore, it is intended that the following claims be interpreted as covering all such alterations and modifications as fall within the true spirit and scope of the disclosure.

What is claimed is:

1. A system, said system comprising:
a memory; and
a processor in communication with said memory, said processor being configured to perform operations, said operations comprising:
training a cr-GAN model with a first data set of a first pharmaceutical and a second data set of a second pharmaceutical;
conditioning said cr-GAN model with at least one conditional variable;
generating, with said cr-GAN model, patient parameters;
replicating a set of patient data of a patient with said patient parameters;
calculating dosage data with said patient parameters based on a therapeutic target; and
displaying said dosage data to a user.

2. The system of claim 1, said operations further comprising:
generating said first data set with a pharmacokinetic model.

3. The system of claim 1, said operations further comprising:
generating said first data set with a quantitative systems pharmacology model.

4. The system of claim 1, said operations further comprising:
describing an interaction between said first pharmaceutical and said patient in said patient data.

5. The system of claim 1, wherein:
said at least one conditional variable includes at least one of the group consisting of baseline tumor size, current tumor size, observed tumor size change, and observed tumor size change rate.

6. The system of claim 1, said operations further comprising:
determining an isobole contour plot based on said dosage data; and
displaying calculated efficacies of said first pharmaceutical and said second pharmaceutical on said isobole contour plot.

7. The system of claim 1, said operations further comprising:
training a second cr-GAN model with a distribution of efficacy measures and tumor sizes associated with said efficacy measures;
sampling from said dosage data with said second cr-GAN model to identify a recommended dosage; and
including said recommended dosage in said dosage data.

8. A computer-implemented method, said method comprising:
training a cr-GAN model with a first data set of a first pharmaceutical and a second data set of a second pharmaceutical;
conditioning said cr-GAN model with at least one conditional variable;
generating, with said cr-GAN model, patient parameters;
replicating a set of patient data of a patient with said patient parameters;
calculating dosage data with said patient parameters based on a therapeutic target; and
displaying said dosage data to a user.

9. The computer-implemented method of claim 8, further comprising:
generating said first data set with a pharmacokinetic model.

10. The computer-implemented method of claim 8, further comprising:
generating said first data set with a quantitative systems pharmacology model.

11. The computer-implemented method of claim 8, further comprising:
describing an interaction between said first pharmaceutical and said patient in said patient data.

12. The computer-implemented method of claim 11, wherein:
said interaction describes at least one of the group consisting of a first absorption, a first distribution, a first metabolism, and a first excretion.

13. The computer-implemented method of claim 8, wherein:
said at least one conditional variable includes at least one of the group consisting of baseline tumor size, current tumor size, observed tumor size change, and observed tumor size change rate.

14. The computer-implemented method of claim 8, further comprising:
determining an isobole contour plot based on said dosage data; and
displaying calculated efficacies of said first pharmaceutical and said second pharmaceutical on said isobole contour plot.

15. The computer-implemented method of claim 8, further comprising:
training a second cr-GAN model with a distribution of efficacy measures and tumor sizes associated with said efficacy measures;
sampling from said dosage data with said second cr-GAN model to identify a recommended dosage; and
including said recommended dosage in said dosage data.

16. A computer program product, said computer program product comprising a computer readable storage medium having program instructions embodied therewith, said program instructions executable by a processor to cause said processor to perform a function, said function comprising:
training a cr-GAN model with a first data set of a first pharmaceutical and a second data set of a second pharmaceutical;
conditioning said cr-GAN model with at least one conditional variable;
generating, with said cr-GAN model, patient parameters;
replicating a set of patient data of a patient with said patient parameters;
calculating dosage data with said patient parameters based on a therapeutic target; and
displaying said dosage data to a user.

17. The computer program product of claim 16, said function further comprising:
generating said first data set with a pharmacokinetic model.

18. The computer program product of claim 16, said function further comprising:
describing an interaction between said first pharmaceutical and said patient in said patient data.

19. The computer program product of claim 16, said function further comprising:
determining an isobole contour plot based on said dosage data; and
displaying calculated efficacies of said first pharmaceutical and said second pharmaceutical on said isobole contour plot.

20. The computer program product of claim 16, said function further comprising:
training a second cr-GAN model with a distribution of efficacy measures and tumor sizes associated with said efficacy measures;
sampling from said dosage data with said second cr-GAN model to identify a recommended dosage; and
including said recommended dosage in said dosage data.
